(19) United States
(12) Patent Application Publication
(10) Pub. No.: US 2012/0283540 A1
(43) Pub. Date: Nov. 8, 2012

(54) IMPLANTABLE SENSOR DEVICE AND MEDICAL DELIVERY DEVICE CONNECTABLE TO SUCH A SENSOR DEVICE

(75) Inventor: Ulrich Brüggemann, Bridgewater, NJ (US)

(73) Assignee: SANOFI-AVENTIS DEUTSCHLAND GMBH, Frankfurt am Main (DE)

(21) Appl. No.: 13/387,862

(22) PCT Filed: Aug. 5, 2010
(86) PCT No.: PCT/EP2010/061420

§ 371 Date: Jul. 26, 2012

(30) Foreign Application Priority Data
Aug. 8, 2009 (EP) 09010271.6

(51) Int. Cl. A61B 5/145

(52) U.S. Cl. 600/365

(57) ABSTRACT

The invention relates to a sensor device for in vivo monitoring of glucose in diabetics, wherein the sensor device comprises a micro-array with multiple sensors to be implanted subcutaneously in a patient wherein each sensor may be activated separately for monitoring purposes. Preferably, the sensors in said micro-array are comprised in a mold that is covered by a metal membrane, and even more preferably, the membrane is electrically openable to expose each specific activated sensor in the micro-array separately for monitoring purposes. A transceiver may be provided for monitoring signals obtained by each specific activated sensor to an external receiver and the receiver may be connected to a display means for displaying data representative of the signals obtained by the specific activated sensor.

### CROSS REFERENCE TO RELATED APPLICATIONS

The present application is a U.S. National Phase Application pursuant to 35 U.S.C. §371 of International Application No. PCT/EP2010/061420 filed Aug. 5, 2010, which claims priority to European Patent Application No. 09010271.6 filed on Aug. 8, 2009. The entire disclosure contents of these applications are herewith incorporated by reference into the present application.

### FIELD OF INVENTION

The present invention is directed to an implantable sensor device, in particular to implantable sensors for in vivo control of glucose in diabetes patients (diabetics), and medical delivery device connectable to such a sensor device.

### BACKGROUND

From EP 0778 897 B1 there are known in vivo enzyme biosensors and more specifically miniature glucose sensors for subcutaneous measurement of glucose in response to the need for frequent or continuous in vivo monitoring of glucose in diabetics, and more particularly a range of possible in vivo glucose electrodes. The desired characteristics of these electrodes include safety, clinical accuracy and reliability, feasibility of in vivo recalibration, stability for at least one hospital shift of eight hours, small size, ease of insertion and removal, and a sufficiently fast response to allow timely intervention.

Furthermore, from CA 2165810 there is known an infusion pump and sensor assembly for delivering medication to a patient, comprising a sensor unit including implantable glucose sensor means for in vivo monitoring of the patient blood glucose parameter, an implantable connector fitting for supporting said sensor means within the patient to permit transcutaneous access to said sensor means for removal and replacement without removing said connector fitting from the patient, control means coupled by said fitting to said sensor means for generating a signal representative of the monitored patient parameter; and pump means for administering medication stored therein to the patient, said pump means including means responsive to said signal to administer the medication in accordance with the monitored patient parameter. The sensor unit comprises a catheter having one end connected to said connector fitting and adapted to extend from said fitting generally to a selected in vivo sensing site within the patient, said sensor means comprising a sensor tip and cable means having a distal end thereof connected to said sensor tip and a proximal end for removable mounting within said connector fitting, said cable means extending from said fitting through said catheter.

US7236812B1 discloses a system, a device and a method for sensing the concentration of an analyte in a fluid (for example, a fluid sample) or matrix. The analyte may be glucose or other chemical of interest. The fluid or matrix may be, for example, the fluid or matrix in the body of an animal (for example, human), or any other suitable fluid or matrix in which it is desired to know the concentration of an analyte. In one embodiment, the system and/or device includes one or more layers having a plurality of analyte-equivalents and mobile or fixed receptor molecules with specific binding sites for the analyte-equivalents and analytes under analysis (for example, glucose). The receptor molecules, when exposed to or in the presence of analyte (that resides, for example, in a fluid in an animal), bind with the analyte (or vice versa). As such, some or all (or substantially all) of the receptor molecules within a given layer may bind with the analyte, which results in a change in the optical properties of one or more of the layers. These layer(s) may be examined or interrogated, via optical techniques, whereby the optical response of the layers and/or, in particular, the substance within the layer(s), may be measured, evaluated and/or analyzed.

Such in vivo enzyme single biosensors have to be replaced frequently as the sensitivity of a sensor tends to be exhausted more or less rapidly and therefore, the lifetime of a sensor is limited. Such replacement tends to be time-consuming, costly and cumbersome as it usually necessitates the patient's consulting of a specialized doctor.

It is therefore an object of the present invention to provide a solution by which the effort for the replacement of such a sensor is reduced.

These objectives are solved according to the invention by the features of the independent claims. Further examples of the invention are described in the subclaims referred back thereto.

The invention provides a sensor device to be implanted subcutaneously in a human body. The sensor device comprises micro-sensors for in-vivo measuring or monitoring of a biological substance or several biological substances or medical-health related targets or the like such as body substances, glucose, vitamins, toxics, metabolites and other ingredients of the body like medical or biological substances or ingredients by which immuno responses can be measured or monitored. A biological substance for the sake of the present invention shall be defined as a particle that is a constituent part of the human or animal body or is linked to the human or an animal body. An example of such a biological substance can be an atom, small organic molecule (e.g. sugar; cholesterol; fatty acid; glucose; pharmaceutically active substance), ion (e.g. Ca2+; Na+; charged protein; buffer component), complex bioorganic molecule (e.g. vitamin; cofactor; hormone), complex polymeric compound (e.g. polynucleotide; protein; receptor, protein hormone, insulin; glucagon; antibody; complex carbohydrate), liquid (e.g. tissue component, blood component), invaded biological particle (virus; bacterium), also toxics or metabolites or other ingredients of the body by which e.g. a disease (e.g. cancer; alcoholism; liver failure; heart failure; kidney failure) or a healthy or defect status can be defined and/or identified or the like. Measuring or monitoring of such a biological substance can be achieved by standard analytic techniques or by particularly adapted or developed techniques. A biological substance can be a mixture of different substances.

With the sensor device the time between implantation of the same and the necessity for removal of the same due to the failure of all micro-sensors is much increased. By this solution relatively simple sensors with a certain lifetime can be used and the frequency of removal from the body can be decreased.

The sensor device to be implanted subcutaneously in a human body according to the invention comprises an arrangement or an array of sensors and in particular micro-sensors, wherein each sensor can be activated separately for monitoring purposes. In this regard, one micro-sensor is activated when another micro-sensor is disabled due to expiration of its lifetime. The sensor device is designed such that one micro-sensor after the other is enabled or activated by a control device. The control device can be part of the senor device which is provided for implantation in the body or can be an external unit which controls the micro-sensors over radio transmission.

Depending on the corresponding application, the sensor device can comprise a big quantity of micro-sensors like for example several hundred micro-sensors, or only a few sensors. In particular, the micro-sensors can be arranged as an array of sensors.

According to an example of the invention, the micro-sensors of the arrangement are disposed in a case which comprises a cover with a plurality of cover parts, wherein each of which is covering a measuring surface of one of the sensors. Therefore, between the measuring surface or the measuring part of each sensor and the periphery or the body area when the sensor device is implanted a cover part is disposed. Each of the cover parts is designed such that it can be removed for activation of the micro-sensor being positioned next to the respective cover part.

According to an example of the invention, each cover part of the micro-sensors is designed such that each cover part at least partly dissolves by the contact with the substance of the body after a predetermined time such that the respective cover part opens the measuring surface of a micro-sensor to the substance of the body, wherein the cover parts of the different micro-sensors are dissolved in sequentially following points of time which correspond to the lifetime of the micro-sensors.

According to another example of the invention, each cover part of the micro-sensors is electrically controllable by the control device such that upon an electrical signal the respective cover part opens the measuring surface of a micro-sensor to the substance of the body.

According to an example of the invention, the case comprises a cover which comprises the cover parts and which extends over at least one side of the case such that it covers the micro-sensors, wherein the material of at least the cover parts of the cover is electrically responsive such that upon a predetermined signal the respective cover part opens the measuring surface of a micro-sensor to the substance of the body.

According to an example of the invention, the sensor device is designed such that in response to an electrical signal which is sent to a cover part, this cover part dissolves or becomes liquid thereby opening the measuring surface to the substance of the body or the body liquid.

According to an example of the invention, the cover of the arrangement of micro-sensors comprises a membrane and in particular a metal membrane or a membrane made of carbon fibres. Further, the metal membrane can be realized such that it is opened electrically to expose each specific activated sensor in the micro-array separately.

The sensor device can be designed such that in response to an electrical signal which is sent to a cover part, this cover part disengages from the cover thereby opening the measuring surface to the substance of the body or the body liquid.

According to an example of the invention, the sensor device comprises an emitter for transmitting sensor signals to an external receiver.

According to an example of the invention, the sensor device comprises a power supply module to which the case can be attached. Thereby, only the case need to be replaced after the expiration of the lifetime of all micro-sensors and the power supply can remain in the human or biological body.

According to another aspect of the invention a measuring system is provided which comprises the sensor device according to the invention and an external monitoring system, wherein the external monitoring system comprises an external receiver for receiving signals form the emitter which is connected to a display means for displaying data representative of the signals obtained by the specific activated sensor.

In this regard, the external receiver can be connected to:
a microprocessor-based comparison and decision-making unit which compares the signals obtained by the specific activated sensor with predetermined blood glucose concentration values, and which, if the result of the comparison is such that certain blood glucose concentration values are exceeded, emits command signals for initiating the administration of a diabetes medicament via a diabetes medicament delivery unit,
a diabetes medicament delivery unit, which upon receipt of said command signal for initiating the administration of a diabetes medicament releases a predetermined dosage of a diabetes medicament.

### BRIEF DESCRIPTION OF THE FIGURES

Without any limitation, the instant invention will, by way of example only, be explained in greater detail below with reference to the drawings in which:

FIG. 1 is a cross-sectional view of an example of the sensor array according to the invention together with an emitter device for signal transmission to an external receiver,

FIG. 2 is a cross-sectional view of the example of the sensor array along the line 2-2 as shown in FIG. 1.

### DETAILED DESCRIPTION

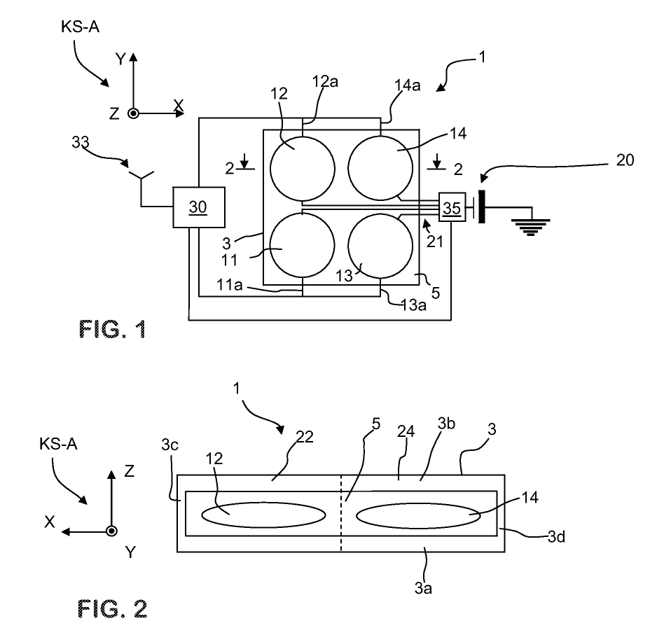

FIG. 1 shows an implantable sensor device 1 according to an example of the present invention. The sensor device 1 comprises a case or housing or mold 3 which can be sealed and in which a substrate or a basic material 5 is integrated. In this basic material an arrangement of micro-sensors 11, 12, 13, 14 is disposed or imbedded. For orientation, a coordinate system KS-A is shown in FIGS. 1 and 2 having the coordinate axes X, Y, Z, wherein the sensors 11, 12, 13, 14 are positioned in the XY-plane.

The sensor device 1 is realized as a miniature sensor device so that it is adapted for implantation in a human body. The sealed housing 3 being is made of a bio-compatible material such as titanium or titanium alloy or plastics. However, other materials can be used.

The arrangement or micro-sensors 11, 12, 13, 14 is disposed in a case 3 or a housing which comprises a cover with a plurality of cover parts 22, 24 (only shown in FIG. 2). Each of which the cover parts 22, 24 is covering a measuring surface of one of the sensors 11, 12, 13, 14. The cover parts are designed such that each of the cover parts can be removed for activation of the micro-sensor being positioned next to the respective cover part. The removal of a cover part can be provided by the material of the cover part which dissolves after a predetermined time for example due to the thickness of the respective cover part. Alternatively or additionally, the cover part can be designed such that it can be removed upon an electrical signal which is sent by the control device to the cover part.

When one cover part is removed, the sensor lying next to the cover part is activated. The sensor can be designed such that it measures or provides a sensor signal as soon as it is in contact with the substance of the biological body in which the sensor device is implanted. Alternatively or additionally, the sensors can be designed such that the respective sensor can be activated by activation of a signal or electrical connection from the control device. Thus, the respective sensor can be activated by sending a corresponding signal from the control device to the respective sensor.

According to an example of the invention, each cover part can be removed due to an electrical signal which is sent from the control device to the respective cover part.

As shown in FIG. 2, the sensors are arranged in a virtual plane of the sensor device 1 and in the form of a matrix. However, other forms of arrangement of the sensors are possible. The number of sensors differs dependent on the respective application. Generally, a plurality of sensors is integrated in the housing 3.

FIG. 2 shows a cross section through a single miniature sensor such as it is comprised in the sensor array shown in FIG. 1.

The structure of the micro-array sensor device 1 according to the invention is such that it provides a multitude of in-vivo sensors 11, 12, 13, 14 which may be individually and systematically activated and used for in-vivo monitoring of a patient's glucose levels. The present invention thus provides for prolonged over-all life time of an implanted sensor and increases the time intervals between a patient's visits to his doctor for replacement of the sensor device 1.

The sensor device 1 comprises a power supply device 20 which may be coupled to the sensors by corresponding connecting lines 21.

The sensors 11, 12, 13, 14 are connected to a control device 30 comprising an emitter device via connecting lines 11a, 12a, 13a, 14a over which each sensor sends signals to the control device 30 which corresponds to the measured state of the liquid of the human body in which the sensor device 1 is implanted. The control device is connected to an antenna 33 in order to transmit the measured signals to an external receiving device. Further, the control device 30 is connected via connecting line 35a to a switch 35 which is coupled to the power supply 20. The control device 20 can command the switch 35 in a way that the power supply 20 is actively connected to one sensor or the sensor array and/or to a cover part which is covering the sensor part or sensor surface of the sensor which lies next to the respective cover part for the removal of the same. The housing 3 comprises a lower membrane or wall 3a and an upper membrane or wall 3b, both extending along the XY-plane. Further, the housing 3 comprises side membranes or side walls 3c, 3d, both extending along the YZ-plane. At least a section of a membrane of the housing is designed such that it allows contact of the liquid of the human body to be measured when a sensor is enabled or actively connected to the power supply 20. For example, the lower and/or upper membrane 3a, 3b, the sensors and the power supply 30 can be designed such that, in the case that one sensor is actively connected to the power supply, at least a section of the lower and/or upper membrane 3a, 3b melts so that the respective sensor gets in contact with the liquid to be measured. This sensor sends signals to the control device 30 which corresponds of the state of the liquid to be measured, for example the concentration of glucose.

Particularly, the sensor device 1 can be designed such that the section of the lower and/or upper membrane 3a, 3b which lies closest to the respective sensor is melting.

Further, the control device 30 includes a function which disables the respective sensor which is actively connected to the power supply at one time. The control device 30 is configures such that the time after which the control respective sensor is disabled corresponds to the life time of the type of sensor used in the sensor array and another sensor is enabled. The order in which the sensors are enabled after the preceding sensor is disabled can be stored in a predetermined manner in the control device 30.

Particularly, the membrane which covers the sensors can be a metal membrane which is opened electrically to expose each specific activated sensor in the micro-array separately.

According to a further example, the control device 30 comprises a transceiver by which the control device can receive signals from an external control device (not shown) like a command to conduct a measurement with one of the sensors arranged in the sensor device 1. The external control device can comprise a radio transmission device or a telemetry unit with a display by which values of the measured state of the liquid in the human body are shown and can be monitored. The external control unit and the sensor device can be configured such that a user or a responsible person can initiate a measurement by the control device based on the values shown on the display.

Further, the external control unit can be configured such that it can be connected to a medical delivery device such as an injection device, an infusion pump, a transplanted delivery device, and/or the like. Generally, the external control device can comprise a transceiver function or only a receiver function or receiver module. In both examples, the external control unit can comprise:
a microprocessor-based comparison and decision-making unit which compares the signals obtained by the specific activated sensor with predetermined blood glucose concentration values, and which, if the result of the comparison is such that certain blood glucose concentration values are exceeded, emits command signals for initiating the administration of a diabetes medicament via diabetes medicament delivery unit,
a diabetes medicament delivery unit, which upon receipt of said command signal for initiating the administration of a diabetes medicament releases a predetermined dosage of a diabetes medicament.

1. Sensor device to be implanted subcutaneously in a human body for in vivo monitoring of a biological substance therein, the sensor device comprising an arrangement of micro-sensors, wherein each micro-sensor can be activated separately for monitoring purposes, characterized in that
the sensor device comprises a control device comprising a control function which is designed to activate micro-sensors of the sensor arrangement and wherein the control function activates one sensor at a point of time at which the control function considers another micro-sensor as being disabled.

2. Sensor device according to claim 1, characterized in that the micro-sensors of the arrangement are disposed in a case which comprises a cover with a plurality of cover parts, wherein each of which is covering a measuring surface of one of the sensors and wherein each of the cover parts is designed such that it can be removed for activation of the micro-sensor being positioned next to the respective cover part.

3. Sensor device according to claim 2, characterized in that each cover part of the micro-sensors is designed such that each cover part at least partly dissolves by the contact with the substance of the body after a predetermined time such that the respective cover part opens the measuring surface of a micro-sensor to the substance of the body, wherein the cover parts of the different micro-sensors are dissolved in sequentially following points of time which correspond to the lifetime of the micro-sensors.

4. Sensor device according to claim 2, characterized in that each cover part of the micro-sensors is electrically controllable such that upon an electrical signal the respective cover part opens the measuring surface of a micro-sensor to the substance of the body.

5. Sensor device according to claim 4, characterized in that the case comprises foil which comprises the cover parts and which extends over at least one side of the case such that it covers the micro-sensors, wherein the material of at least the cover parts of the foil is electrically responsive such that upon a predetermined signal the respective cover part opens the measuring surface of a micro-sensor to the substance of the body.

6. Sensor device according to claim 5, characterized in that in response to an electrical signal which is sent to a cover part, this cover part dissolves or becomes liquid thereby opening the measuring surface to the substance of the body.

7. Sensor device according to claim 5, characterized in that in response to an electrical signal which is sent to a cover part, this cover part disengages from the cover thereby opening the measuring surface to the substance of the body.

8. Sensor device according to claim 1, characterized in that the sensor device comprises an emitter for transmitting sensor signal to an external receiver.

9. Sensor device according to claim 2, characterized in that the sensor device comprises a power supply module to which the case can be attached.

10. Measuring system comprising the sensor device according to claim 1 and an external monitoring system, characterized in that the external monitoring system comprises an external receiver for receiving signals form the emitter which is connected to a display means for displaying data representative of the signals obtained by the specific activated sensor.

11. A medical delivery device connectable to a sensor device according to claim 10, wherein the external receiver is connected to:
a microprocessor-based comparison and decision-making unit which compares the signals obtained by the specific activated sensor with predetermined blood glucose concentration values, and which, if the result of the comparison is such that certain blood glucose concentration values are exceeded, emits command signals for initiating the administration of a diabetes medicament via diabetes medicament delivery unit,
a diabetes medicament delivery unit, which upon receipt of said command signal for initiating the administration of a diabetes medicament releases a predetermined dosage of a diabetes medicament.
